(19) United States
(12) Patent Application Publication
(10) Pub. No.: US 2007/0123480 A1
(43) Pub. Date: May 31, 2007

(54) Oligonucleotides targeting prion diseases

(75) Inventors: Jean-Marc Juteau, Blainville (CA); Andrew Vaillant, Roxboro (CA)

Correspondence Address: BIRCH STEWART KOLASCH & BIRCH, PO BOX 747, FALLS CHURCH, VA, 22040-0747, US

(73) Assignee: Replicor Inc., Laval, QC (CA)

(21) Appl. No.: 10/571,172

(22) PCT Filed: Sep. 10, 2004
(86) PCT No.: PCT/IB04/03740

§ 371 Date: Oct. 10, 2006

Related U.S. Application Data
(60) Provisional application No. 60/584,627, filed on Jun. 30, 2004.

(30) Foreign Application Priority Data
Sep. 11, 2003 (IB) PCT/IB03/04573

(51) Int. Cl. A61K 48/00

(52) U.S. Cl. 514/44; 514/81

(57) ABSTRACT

Randomer phosphorothioate oligonucleotide compositions have been described that inhibit PrPc conversion to PrPcs with a high level of potency. Pharmaceutical compositions or kits containing such compounds, and methods of using such compounds in the treatment, control, or prevention of prion diseases are also described.

### BACKGROUND OF THE INVENTION

The present invention concerns treatment or prevention of transmissible spongiform encephalopathies, also referred to as prion diseases.

Transmissible spongiform encephalopathies (TSEs) encompass a group of potentially fatal neurodegenerative diseases in animals and humans. The etiology of naturally occurring TSEs seems to include horizontal and vertical transmission as well as genetic predisposition, yet for the majority of cases the etiology is unclear. The onset of clinical illness is preceded by a prolonged incubation period of months to decades. Clinical symptoms of TSEs include dementia and loss of movement and coordination. Neuropathological examination in disease cases typically reveals gliosis and the presence of spongiform encaphalophy, sometimes accompanied by the formation of amyloid deposits (amyloid plaques).

TSEs, which include Creutzfeldt-Jacob Disease (CJD), variant CJD (vCJD), fatal familial insomnia (FFI), Gerstmann-Straussler-Scheinker Disease (GSS), kuru, bovine spongiform-encephalopathy (BSE), feline spongiform encephalopathy (FSE), transmissible mink encephalopathy (TME), chronic wasting disease (CMD), and scrapie, are characterized by the accumulation of aggregates of the abnormal prion protein (PrPsc) in the brain and other infected tissues. The normal form, PrPc, which is dominated by alpha-helices towards the C-terminus, is most abundant in the central nervous system but its physiological function is unknown. The accumulation of the beta-structure rich isoform, PrPsc, is widely believed to result from the ability of this isoform to stabilize thermodynamically, similarly folded forms during the folding of cellular PrPc. This process contributes to the formation of increasing numbers of misfolded prion proteins which upon aggregation, form the major component of amyloid plaques characteristic of TSE's.

Caughey and coworkers (1993) tested sulfated polyanions as inhibitors of scrapie-associated PrPsc accumulation in cultured cells. Pentosan polysulfate and the amyloid-binding dye Congo red potently inhibited the accumulation of PrPsc in cells without apparent effects on the metabolism of the normal isoform PrPc. A comparision of the activity of pentosan polysulfate with that of sulfated glycans, non-sulfated polyanions, dextran and DEAE-dextran has suggested that the density of sulfation and molecular size are factors influencing anti-PrPsc activity of sulfated polyanions. Shyng and coworkers (1995) also reported that pentosan polysulfate and related compounds rapidly and dramatically reduced the amount of PrPc, the non-infectious precursor of PrPsc, present on the cell surface.

Another study reported that treatment of TSE-infected animals with certain cyclic tetrapyrroles (porphyrins and phthalocyanines) increased survival time from 50 to 300%. The significant inhibition of TSE disease by structurally dissimilar tetrapyrroles identifies these compounds as anti-TSE drugs (Priola et al., 2000).

Supatappone and coworkers (2001) demonstrated that exposure of scrapie-infected neuroblastoma cells to 3 micrograms of branched polyamines, including polyamidoamine and polypropyleneimine, for 4 weeks not only reduced PrPsc to a level undetectable by Western blot but also eradicated prion infectivity as determined by a bioassay in mice. The activity of branched polyamines in vitro was prion strain dependent.

Ampliotericine B (AmB), a macrolide polyene antibiotic, is one of the few drugs that has shown therapeutic activity in scrapie-infected hamsters. A study showed that treatment with an AmB derivative delayed the progression of the disease, possibly by preventing the replication of the scrapie protein at the inoculation site where the cells appear to be the first producing abnormal PrP (Grigoriev et al. 2002)

Poli and collaborators (2003) demonstrated the ability of synthesized Congo red derivatives to prevent the prion protein conversion in cell-free and cellular assays. However, the most active compound in the cellular assay was also highly toxic at the effective dose.

Another study reported that heparan sulfate mimetics could abolish prion propagation in scrapie-infected cells. PrPsc does not reappear for up to 50 days post-treatment. When tested in vivo, one compound hampered PrPsc accumulation in scrapie- and BSE-infected mice and prolonged significantly the survival time of scrapie-infected hamsters (Adjou et al. 2003).

Kocisko and coworkers (2003) are reported to have identified new inhibitors of PrPsc formation from a library of compounds. Several classes of compounds were represented in the 17 most potent inhibitors, including naturally occurring polyphenols (e.g., tannic acid and tea extracts), phenothiazines, antihistamines, statins, and antimalarial compounds.

Quinacrine was shown to hamper de novo generation of fibrillogenic prion protein. However, in vivo, no detectable effect was observed in an animal model, consistent with other recent studies and preliminary observations in humans. Despite its ability to cross the blood-brain barrier, the use of quinacrine for the treatment of CJD is questionable (Barret et al. 2003) (Nakajirna et al, 2004).

The therapeutic efficacy of direct drug infusion into the brain was assessed in transgenic mice intracerebrally infected with the scrapie agent. Pentosan polysulfate (PPS) gave the most dramatic prolongation of the incubation period, and AmB had intermediate effects, but antimalarial drugs such as quinacrine gave no significant prolongation. However, at doses higher than that providing the maximal effects, intraventricular PPS infusion caused adverse effects such as hematoma formation in the experimental animals (Doh-ura et al., 2004).

The squalene synthase inhibitor squalestatin reduced the cholesterol content of cells and prevented the accumulation of PrPsc in three prion-infected cell lines. Cells treated with squalestatin were also protected against microglia-mediated killing. These effects of squalestatin were dose-dependent and were evident at nanomolar concentrations (Bate et al., 2004).

In a review article, Koster et al. (2003) described a number of possible therapeutic agents that have been tried and some reported to have activity against TSEs but most of these compounds have limitations in terms of toxicity and pharmacokinetics. Congo red, anthracyclines, and the polyanion dextran sulfate have limited ability to cross the blood-brain barrier and may be toxic. The efficacy of polyene antibiotics seems to be restricted to certain scrapie strains. Tetrapyrroles and tetracyclines with low toxicities and favorable pharmacokinetics could be useful in preventing PrPsc accumulation. Compounds like branched polyamnines, Cp-60, analogs of Congo red, quinacrine and chlorpromazine, beta-sheet breaker peptides and inhibitory peptides, active immunization using recombinant PrP and passive immunization with anti-PrP antibodies, have potential use as therapeutic agents but will need further research and clinical trials.

There is no currently available treatment to cure or prevent the development of transmissible spongiform encephalopathies and other prion-associated diseases. There is also no treatment for animal or human tissue products to prevent transmission of prion diseases. It would be useful to have compounds, methods of treatment, and formulations to treat, prevent transmission and development of and reverse progression in prion diseases.

Approximately 80 million units of blood are donated annually worldwide (World Health Organization, 2004). There have been chronic shortages of blood, partly because of increased demand from modem surgical techniques. For example, people who are undergoing aggressive cancer chemotherapy treatments require blood transfusions because their own body's ability to make blood cells diminishes. Premature infants may require blood transfusions to carry oxygen throughout their bodies. Medical treatments, such as organ transplants and cardiac bypass surgery, that require a large amount of blood, were uncommon 30 years ago, yet today are routine. And the aging of the population means that more people live longer and are more likely to need medical treatments that require safe blood and blood products. Blood supplies are tested for several infectious agents and are treated for such agents when treatments are available. But no treatments are currently available to safely inactivate or destroy prions in blood and blood product supplies without affecting the required properties of such biological products.

The information provided and references cited herein is intended only to assist the understanding of the reader, and does not constitute an admission that any of the information or references constitutes prior art to the present invention.

### SUMMARY OF THE INVENTION

The present invention concerns oligonucleotides that have anti-prion activity, and thus can be used in treatment, control, or prevention of one or more prion diseases. Likewise, such oligonucleotides can be used to treat biological materials, e.g., to prevent or reduce the chance of infection following use of the biological material.

In addition, the inventors discovered that different length oligonucleotides have varying anti-prion effect, and further that the length of anti-prion oligonucleotide that produces potent anti-prion effect is usually about 40 nucleotides or longer, e.g., in the range of 40-120 nucleotides. In view of the present discoveries concerning anti-prion properties of oligonucleotides, this invention provides oligonucleotide anti-prion agents that can have activity against several different prion disease agents, and can even be selected as broad-spectrum anti-prion agents. Such anti-prion agents are particularly advantageous in view of the limited anti-prion therapeutic options currently available.

Therefore, the oligonucleotides of the present invention are useful in therapy for treating or preventing prion diseases and in treating or preventing other diseases whose etiology is prion-based.

Thus, the invention concerns anti-prion oligonucleotides and oligonucletide formulations that includes at least one anti-prion oligonucleotide, e.g., at least 6 nucleotides in length, adapted for use as an anti-prion agent. Preferably the anti-prion activity of the oligonucleotide occurs principally by a sequence independent mode of action. Such a formulation can include a mix of different oligonucleotides, e.g., at least 2, 3, 5, 10, 50, 100, or even more.

A related aspect concerns an anti-prion oligonucleotide randomer formulation, where the anti-prion activity of the randomer occurs principally by a sequence independent mode of action. Such a randomer formulation can, for example, include a mixture of randomers of different lengths, e.g., at least 2, 3, 5, 10, or more different lengths.

In another aspect, the invention provides an oligonucleotide having anti-prion activity against a prion disease, where the oligonucleotide is at least 29 nucleotides in length (or in particular embodiments, at least 30, 32, 34, 36, 38, 40, 46, 50, 60, 70, 80, 90, 100, 110, or 120 nucleotides in length). In particular embodiments, the sequence of the oligonucleotide is not complementary to any portion of the genome sequence of the aniimal subject to the particular prion disease of interest.

In another aspect, the invention provides an oligonucleotide formulation, containing at least one oligonucleotide having anti-prion activity against a prion disease, where the oligonucleotide is at least 6 nucleotides in length (in particular embodiments, at least 10, 15, 18, 20, 22, 24, 26, 28, 29, 30, 32, 34, 36, 38, 40, 46, 50, 60, 70, 80, 90, 100, 110, or 120 nucleotides in length). In certain embodiments, the sequence of the oligonucleotide is less than 70% complementary to any portion of the genomic nucleic acid sequence of the subject aniimal for the particular prion disease and does not consist essentially of polyA, polyC, polyG, polyT, Gquartet, or a TG-rich sequence. In particular embodiments, the oligonucleotide has less than 65%, 60%, 55%, 50%, 80% 90%, 95%, or 100% complementarity to any portion of the genomic nucleic acid sequence of the animal subject to, the particular prion disease.

Related aspects concern isolated, purified or enriched anti-prion oligonucleotides as described herein, e.g., as described for anti-prion oligonucleotide formulations, as well as other oligonucleotide preparations, e.g., preparations suitable for in vivo use.

Anti-Prion oligonucleotides useful in the present invention can be of various lengths, e.g., at least 6, 10, 14, 15, 20, 25, 28, 29, 30, 35, 38, 40, 46, 50, 60, 70, 80, 90, 100, 110, 120, 140, 160, or more nucleotides in length. Likewise, the oligonucleotide can be in a range, e.g., a range defined by taking any two of the preceding listed values as inclusive end points of the range, for example 10-20, 20-40, 30-50, 40-60, 40-80, 60-120, and 80-120 nucleotides. In particular embodiments, a minimum length or length range is combined with any other of the oligonucleotide specifications listed herein for the present anti-prion oligonucleotides.

The anti-prion nucleotide can include various modifications, e.g., stabilizing modifications, and thus can include at least one modification in the phosphodiester linkage and/or on the sugar, and/or on the base. For example, the oligonucleotide can include one or more phosphorothioate linkages, phosphorodithioate linkages, and/or methylphosphonate linkages; modifications at the 2′-position of the sugar, such as 2′-O-methyl modifications, 2′-amino modifications, 2′-halo modifications such as 2′-fluoro; acyclic nucleotide analogs, and can also include at least one phosphodiester linkage. Other modifications are also known in the art and can be used. In oligos that contain 2′-O-methyl modifications, the oligo should not have 2′-O-methyl modifications throughout, as current results suggest that such oligos do not have suitable activity. In particular embodiments, the oligonucleotide has modified linkages throughout, e.g., phosphorothioate; has a 3′- and/or 5′-cap; includes a terminal 3′-5′ linkage; the oligonucleotide is or includes a concatemer consisting of two or more oligonucleotide sequences joined by a linker(s)

In particular embodiments, the oligonucleotide binds to one or more PrP proteins; the sequence of the oligonucleotide (or a portion thereof, e.g., at least ½) is derived from a genome of a subject animal; the activity of an oligonucleotide with a sequence derived from a genome of a subject animal is not superior to a randomer oligonucleotide or a random oligonucleotide of the same length; the oligonucleotide includes a portion complementary to a genome of a subject animal and a portion not complementary to a genome of a subject animal; the sequence of the oligonucleotide is derived from a PrP sequence; unless otherwise indicated, the sequence of the oligonucleotide includes A(x), C(x), G(x), T(x), AC(x), AG(x), AT(x), CG(x), CT(x), or GT(x), where x is 2, 3, 4, 5, 6, . . . 60 . . . 120 . . . ; the oligonucleotide is single stranded (RNA or DNA); the oligonucleotide is double stranded (RNA or DNA); the oligonucleotide includes at least one Gquartet or CpG portion; the oligonucleotide includes a portion complementary to a mRNA of a subject animal; the oligonucleotide includes at least one non-Watson-Crick oligonucleotide and/or at least one nucleotide that participates in non-Watson-Crick binding with another nucleotide; the oligonucleotide is a random oligonucleotide, the oligonucleotide is a randomer or includes a randomer portion, e.g., a randomer portion that has a length as specified above for oligonucleotide length; the oligonucleotide is linked or conjugated at one or more nucleotide residues to a molecule that modifies the characteristics of the oligonucleotide, e.g. to provide higher stability (such as stability in serum or stability in a particular solution), lower serum interaction, higher cellular uptake, improved ability to be formulated for delivery, a detectable signal, improved pharmacokinetic properties, specific tissue distribution, and/or lower toxicity.

Oligonucleotides can also be used in combinations, e.g., as a mixture. Such combinations or mixtures can include, for example, at least 2, 4, 10, 100, 1000, 10000, 100,000, 1,000,000, or more different oligonucleotides. Such combinations or mixtures can, for example, be different sequences and/or different lengths and/or different modifications and/or different linked or conjugated molecules. In particular embodiments of such combinations or mixtures, a plurality of oligonucleotides have a minimum length or are in a length range as specified above for oligonucleotides. In particular embodiments of such combinations or mixtures, at least one, a plurality, or each of the oligonucleotides can have any of the other properties specified herein for individual anti-prion oligonucleoties (which can also be in any consistent combination).

The invention also provides an anti-prion pharmaceutical composition that includes a therapeutically effective amount of a pharmacologically acceptable, anti-prion oligonucleotide at least 6 nucleotides in length (or other length as listed herein), and a pharmaceutically acceptable carrier. Preferably the anti-prion activity of the oligonucleotide occurs principally by a sequence independent mode of action. In particular embodiments, the oligonucleotide or a combination or mixture of oligonucleotides is as specified above for individual oligonucleotides or combinations or mixtures of oligonucleotides. In particular embodiments, the pharmaceutical compositions are approved for administration to a human, or a non-human animal such as a non-human mammal.

In particular embodiments, the pharmaceutical composition is adapted for the treatment, control, or prevention of a disease with a prion etiology; adapted for treatment, control, or prevention of a prion disease; is adapted for delivery by intraocular administration, oral ingestion, enteric administration, inhalation, cutaneous, subcutaneous, intramuscular, intraperitoneal, intrathecal, intracerebral, intratracheal, or intravenous injection, or topical administration. In particular embodiments, the composition includes a delivery system, e.g., targeted to specific cells or tissues; a liposomal formulation, a penetration enhancer, a surfactant, another anti-prion drug, e.g., a non-nucleotide anti-prion polymer, an antisense molecule, an siRNA, or a small molecule drug.

In particular embodiments, the anti-prion oligonucleotide, oligonucleotide preparation, oligonucleotide formulation, or anti-prion pharmaceutical composition has an IC50 for a prion target (e.g., any of particular prion disease as indicated herein) of 1.0, 0.50, 0.20, 0.10, 0.09. 0.08, 0.07, 0.75, 0.06, 0.05, 0.045, 0.04, 0.035, 0.03, 0.025, 0.02, 0.015, or 0.01 μM or less.

In particular embodiments of formulations, pharmaceutical compositions, and methods for prophylaxis or treatment, the composition or formulation is adapted for treatment, control, or prevention of a disease with prion etiology; is adapted for the treatment, control or prevention of a prion disease; is adapted for delivery by a mode selected from the group consisting of intraocular, oral ingestion, enterally, inhalation, or cutaneous, subcutaneous, intramuscular, intraperitoneal, intrathecal, intracerebral, intratracheal, intraventricular, intracranial, topical or intravenous injection delivery; fuirther comprises a delivery system, which can include or be associated with a molecule increasing affinity with specific cells; further comprises at least one other anti-prion drug in combination (e.g., pentosan polysulfate); and/or further comprises an anti-prion polymer in combination.

In another aspect, the invention provides a kit that includes at least one anti-prion oligonucleotide or oligonucleotide formulation in a labeled package, where the anti-prion activity of the oligonucleotide occurs principally by a sequence independent mode of action and the label on the package indicates that the anti-prion oligonucleotide can be used against at least one prion disease.

In particular embodiments the kit includes a pharmaceutical composition that includes at least one anti-prion oligonucletide as described herein; the anti-prion oligonucleotide is adapted for in vivo use in an animal and/or the label indicates that the oligonucleotide or composition is acceptable and/or approved for use in an animal; the animal is a mammal, such as human, or a non-human mammal such as bovine, porcine, a rumiant, ovine, or equine; the animal is a non-human animal; the kit is approved by a regulatory agency such as the U.S. Food and Drug Administration or equivalent agency.for use in an animal, e.g., a human; the kit is approved by the U.S. Food and Drug Administration or equivalent regulatory agency for an anti-prion indication; the kit includes written instructions for administration to a subject for an anti-prion indication.

In another aspect, the invention provides a method for selecting an anti-prion oligonucleotide, e.g, a sequence independent anti-prion oligonucleotide, for use as an anti-prion agent. The method involves synthesizing a plurality of different random oligonucleotides, testing the oligonucleotides for activity in inhibiting the ability of a PrP to alter to PrPsc and/or to aggregate, and selecting an oligonucleotide having a pharmaceutically acceptable level of activity for use as an anti-prion agent.

In particular embodiments, the different random oligonucleotides comprises randomers of different lengths; the random oligonucleotides can have different sequences or can have sequence in common, such as the sequence of the shortest oligos of the plurality; and/or the different random oligonucleotides comprise a plurality of oligonucleotides comprising a randomer segment at least 5 nucleotides in length or the different random oligonucleotides include a plurality of randomers of different lengths. Other oligonucleotides, e.g., as described herein for anti-prion oligonucleotides, can be tested in a particular system.

In yet another aspect, the invention provides a method for the prophylaxis or treatment of a prion disease in a subject by administering to a subject in need of such treatment a therapeutically effective amount of at least one pharmacologically acceptable oligonucleotide as described herein, e.g., a sequence independent oligonucleotide at least 6 nucleotides in length, or an anti-prion pharmaceutical composition or formulation containing such oligonucleotide. In particular embodiments, the prion disease can be any of those listed herein; the subject is a type of subject as indicated herein, e.g., human, non-human animal, non-human mammal, bovine, porcine, a ruminant, ovine, or equine; the treatment is for a prion disease or disease with a prion etiology.

In particular embodimnents, an anti-prion oligonucleotide (or oligonucleotide formulation or pharmaceutical composition) as described herein is administered; administration is a method as described herein; a delivery system or method as described herein is used.

In another aspect, the discovery that non-sequence dependent interactions produce effective anti-prion activity provides a method of screening to identify a compound that alters formation of PrPsc, e.g., binding of an oligonucleotide to a PrP. For example, the method can involve determining whether a test compound reduces the binding of oligonucleotide to PrP.

In particular embodiments, any of a variety of assay formats and detection methods could be used to identify such alteration (e.g., alteration in binding), e.g., by contacting the oligonucleotide with the PrP (or cell in a cell-based assay) in the presence and absence of a compound(s) to be screened (e.g., in separate reactions) and determining whether a difference occurs in binding of the oligo to PrP (or formation of PrPsc) in the presence of the compound compared to the absence of the compound. The presence of such a difference is indicative that the compound alters the binding of the random oligonucleotide to the PrP (or formation of PrPsc). Alternatively, a competitive displacement can be used, such that oligonucleotide is bound to the PrP and displacement by added test compound is determined, or conversely test compound is bound and displacement by added oligonucleotide is determined.

In particular embodiments, the oligonucleotide is as described herein for anti-prion oligonucleotides; the oligonucleotide is at least 6, 8, 10, 15, 20, 25, 29, 30, 32, 34, 36, 38, 40, 46, 50, 60, 70, 80, 90, 100, 110, or 120 nucleotides in length or at least another length specified herein for the anti-prion oligonucleotides, or is in a range defined by taking any two of the preceding values as inclusive endpoints of the range; the test compound(s) is a small molecule; the test compound has a molecular weight of less than 400, 500, 600, 800, 1000, 1500, 2000, 2500, or 3000 daltons, or is in a range defined by taking any two of the preceding values as inclusive endpoints of the range; at least 100, 1000, 10,000, 20,000, 50,000, or 100,000 compounds are screened; the oligonucleotide has an IC50 of equal to or less than 1.0, 0.500, 0.200, 0.100, 0.075, 0.05, 0.045, 0.04, 0.035, 0.03, 0.025, 0.02, 0.015, or 0.01 μM.

In a related aspect, the invention provides an anti-prion compound identified by the preceding method, e.g., a novel anti-prion compound.

In a further aspect, the invention provides a method for purifying oligonucleotides binding to at least PrP from a pool of oligonucleotides by contacting the pool with at least PrP, e.g., bound to a stationary phase medium, and collecting oligonucleotides that bind to the PrP(s). Generally, the collecting involves displacing the oligonucleotides from the PrP(s). The method can also involve sequencing and/or testing anti-prion activity of collected oligonucleotides (i.e., oligonucleotides that bound to PrP).

In particular embodiments, the bound oligonucleotides of the pool are displaced from the stationary phase medium by any appropriate method, e.g., using an ionic displacer, and displaced oligonucleotides are collected. Typically for the various methods of displacement, the displacement can be performed in increasing stringent manner (e.g., with an increasing concentration of displacing agent, such as a salt concentration, so that there is a stepped or continuous gradient), such that oligonucleotides are displaced generally in order of increased binding affinity. In many cases, a low stringency wash will be performed to remove weakly bound oligonucleotides, and one or more fractions will be collected containing displaced, tighter binding oligonucleotides. In some cases, it will be desired to select fractions that contain very tightly binding oligonucleotides (e.g., oligonucleotides in fractions resulting from displacement by the more stringent displacement conditions) for further use.

Similarly, the invention provides a method for enriching oligonucleotides from a pool of oligonucleotides binding to at least one PrP, by contacting the pool with one or more PrP's, and amplifying oligonucleotides bound to the PrPs to provide an enriched oligonucleotide pool. The contacting and amplifying can be performed in multiple rounds, e.g., at least 1, 2, 3, 4, 5, 10, or more additional times using the enriched oligonucleotide pool from the preceding round as the pool of ohgonucleotides for the next round. The method can also involve sequencing and testing anti-prion activity of oligonucleotides in the enriched oligonucleotide pool following one or more rounds of contacting and amplifying.

The method can involve displacing oligonucleotides from the PrP with any of a variety of techniques, such as those described above, e.g., using a displacement agent. As indicated above, it can be advantageous to select the tighter binding oligonucleotides for further use, e.g., in further rounds of binding and amplifying. The method can further involve selecting one or more enriched oligonucleotides, e.g., high affinity oligonucleotides, for further use. In particular embodiments, the selection can include eliminating oligonucleotides that have sequences complementary to subject animal mRNA or genomic sequences for a particular prion disease of interest. Such elimination can involve comparing the oligonucleotide sequence(s) with sequences from the particular host in a sequence database(s), e.g., using a sequence alignment program (e.g., a BLAST search), and eliminating those oligonucleotides that have sequences identical or with a particular level of identity to a host sequence. Eliminating such host complementary sequences and/or selecting one or more oligonucleotides that are not complementary to host sequences can also be done for the other aspects of the present invention.

In the preceding methods for identifying, purifying, or enriching oligonucleotides, the oligonucleotides can be of types as described herein. The above methods are advantageous for identifying, purifying or enriching high affinity oligonucleotides, e.g., from an oligonucleotide randomer preparation.

In a related aspect, the invention concerns an anti-prion oligonucleotide preparation that includes one or more oligonucleotides identified using a method of any of the preceding methods for identifying, obtaining, or purifying anti-prion oligonucleotides from an initial oligonucleotide pool, where the oligonucleotides in the oligonucleotide preparation exhibit higher mean binding affinity with one or more PrP's than the mean binding affinity of oligonucletides in the initial oligonucleotide pool.

In particular embodiments, the mean binding affinity of the oligonucleotides is at least two-fold, 3-fold, 5-fold, 10-fold, 20-fold, 50-fold, or 100-fold greater than the mean binding affinity of oligonucleotides in the initial oligonucleotide pool, or even more; the median of binding affinity is at least two-fold, 3-fold, 5-fold, 10-fold, 20-fold, 50-fold, or 100-fold greater relative to the median of the binding affinity of the initial oligo pool, where median refers to the middle value.

In yet another aspect, the invention provides an anti-prion polymer mix that includes at least one anti-prion oligonucleotide and at least one non-nucleotide anti-prion polymer. In particular embodiments, the oligonucleotide is as described herein for anti-prion oligonucleotides and/or the anti-prion polymer is as described herein or otherwise known in the art or subsequently identified.

In yet another aspect, the invention provides an oligonucleotide randomer, where the randomer is at least 6 nucleotides in length. In particular embodiments the randomer has a length as specified above for anti-prion oligonucleotides; the randomer includes at least one phosphorothioate linkage; the randomer includes at least 50% phosphorothioate linkages; the randomer includes at least 80% phosphorothioate linkages; the randomer includes all phosphorothioate linkages; the randomer includes at least one phosphorodithioate linkage or other modification as listed herein; the randomer includes at least 20, 30, 40, 50, 60, 70, 80, or 90% modified linkages (e.g., of a type specified herein such as phosphorothioate or phosphorodithioate); the randomer oligonucleotides include at least one non-randomer segment (such as a segment complementary to a selected subject animal nucleic acid sequence), which can have a length as specified above for oligonucleotides; the randomer is in a preparation or pool of preparations containing at least 5, 10, 15, 20, 50, 100, 200, 500, or 700 micromol, 1, 5, 7, 10, 20, 50, 100, 200, 500, or 700 immol, or 1 mole of randomer, or a range defined by taking any two different values from the preceding as inclusive end points, or is synthesized at one of the listed scales or scale ranges.

Likewise, the invention provides a method for preparing anti-prion randomers, by synthesizing at least one randomer, e.g., a randomer as described above.

In yet another aspect, the invention provides a method for reducing prion activity in a biological material in vitro, by contacting the biological material with at least one anti-prion oligonucleotide, e.g., an anti-prion nucleotide as described herein.

In particular embodiments, the biological material is animal blood (e.g., human, bovine, or ovine blood); the biological materials is an animal blood product (e.g., human, bovine, or ovine blood product); the biological material is a mammalian tissue (e.g., human, bovine, or ovine tissue); the biological material is a mammalian organ (e.g., human, bovine, or ovine organ).

In connection with modifying characteristics of an oligonucleotide by linking or conjugating with another molecule or moiety, the modifications in the characteristics are evaluated relative to the same oligonucleotide without the linked or conjugated molecule or moiety.

In the context of the present invention, unless specifically limited the term “oligonucleotide (ON)” means oligodeoxynucleotide (ODN) or oligodeoxyribonucleotide or oligoribonucleotide. Thus, “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent intemucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for a protein target and increased stability in the presence of nucleases. Examples of modifications that can be used are described herein. Oligonucleotides that include backbone and/or other modifications can also be referred to as oligonucleosides.

The terms “prion”, “prion protein”, “infectious protein” and the like are used interchangeably herein to refer to the infectious PrPsc form of a PrP protein, and is a contraction of the words “protein” and “infection”. Particles are comprised largely, if not exclusively, of PrPsc molecules encoded by a PrP gene. Prions are distinct from bacteria, viruses and viroids. Known prions infect animals to cause scrapie, a transmissible, degenerative disease of the nervous system of sheep and goats, as well as bovine spongiform encephalopathy (BSE), or “mad cow disease,” and feline spongiform encephalopathy in cats. Four prion diseases known to affect humans are: (1) kuru, (2) Creutzfeldt-Jakob Disease (CJD), (3) Gerstmann-Straussler-Scheinker Disease (GSS), and (4) fatal familian insomnia (FFI) (also referred to as fatal insomnia (FI)). Variant CJD (vCJD) is also known, and is related to human ingestion of material from animals infected with BSE.

As used herein “prion” includes all forms of prions causing all or any of these diseases or other diseases of similar pathology in any animals and in particular in humans and domesticated farm animals.

The terms “PrP protein”, “PrP” and like are used interchangeably herein and shall mean both the infectious particle form PrPsc known to cause diseases (spongiform encephalopathies) in humans and animals and the noninfectious form PrPc which, under appropriate conditions is converted to the infectious PrPsc form.

The term “PrP gene” is used herein to describe genetic material which encodes PrP proteins including those with polymorphisms and pathogenic mutations (a number of which are known). The term “PrP gene” refers generally to any gene of any species which encodes any form of a prion protein.

As used herein in connection with anti-prion action of a material, the phrase “sequence independent mode of action” indicates that the mechanism by which the material exhibits an anti-prion effect is not due to hybridization of complementary nucleic acid sequences, e.g., an antisense effect. Furthermore, this term also implies that the mechanism of action is not due to a sequence dependent aptamer interaction with prion proteins. Conversely, a “sequence dependent mode of action” means that the anti-prion effect of a material involves hybridization of complementary nucleic acid sequences or the specific binding of a nucleic acid derived from its specific sequence. It also describes a sequence specific aptameric interaction between a nucleic acid sequence and a protein.

As used herein in connection with oligonucleotides or other materials, the term “anti-prion” refers to an effect which occurs in the presence of oligonucleotides or other agents which inhibit prion diseases by reducing or inhibiting the conversion of PrPc to PrPsc and/or reducing or inhibiting the accumulation of intracellular PrP or PrPsc and/or PrPsc aggregation into amyloid plaques and/or reducing the internalization of prion protein and/or reducing or inhibiting cell death induced by conversion of PrP or accumulation of PrPsc.

The term “anti-prion oligonucleotide formulation” refers to a preparation that includes at least one anti-prion oligonucleotide that is adapted for use as an anti-prion agent. The formulation includes the oligonucleotide or oligonucleotides, and can contain other materials that do not interfere with use of this oligonucleotide as an anti-prion agent in vivo. Such other materials can include without restriction diluents, excipients, carrier materials, delivery systems and/or other anti-prion materials.

As used herein, the term “pharmaceutical composition” refers to an anti-prion oligonucleotide formulation that includes a physiologically or pharmaceutically acceptable carrier or excipient. Such compositions can also include other components that do not make the composition unsuitable for administration to a desired subject, e.g., a human. Typically the composition is sufficiently sterile to be acceptable to a reasonable medical practitioner for administration to a human subject.

As used in connection with an anti-prion formulation, pharmaceutical composition, or other material, the phrase “adapted for use as an anti-prion agent” indicates that the material exhibits an anti-prion effect and does not include any component or material that makes it unsuitable for use in inhibiting a prion-associated disease in an in vivo system, e.g., for administering to a subject such as a human subject.

As used herein in connection with administration of an anti-prion material, the term “subject” refers to a living higher organism, including, for example, animals such as mammals, e.g., humans, non-human-primates, bovines, porcines, ovines, equines, canines, felines and birds.

In the present application, the term “randomer” is intended to mean a single stranded DNA having a wobble (N) at every position, such as NNNNNNNNNN. Each base is synthesized as a wobble such that this ON actually exists as a population of different randomly generated sequences of the same size.

As used herein in connection with oligonucleotide sequences, the term “random” characterizes a sequence or an ON that is not complementary to a MRNA of the animal subject to the particular prion disease of interest, and which is selected to not form hairpins and not to have palindromic sequences contained therein. When the term “random” is used in the context of anti-prion activity of an oligonucleotide toward a particular prion disease, it implies the absence of complementarity to a MRNA of animals subject to that particular prion disease.

The phrase “derived from a genome of a subject animal” indicates that aparticular sequence has a nucleotide base sequence that has at least 85% identity to a nucleotide sequence of an animal subject to the particular prion disease, or, its complement, or is a corresponding RNA sequence. In particular embodiments, the identity is at least 90, 95, 98, 99, or 100%.

As used herein, the term “delivery system” refers to a component or components that, when combined with-an oligonucleotide as described herein, increases the amount of the oligonucleotide that contacts the intended location in vivo, and/or extends the duration of its presence at the target, e.g., by at least 10, 20, 50, or 100%, or even more as compared to the amount and/or duration in the absence of the delivery system, and/or prevents or reduces interactions that cause side effects.

The term “therapeutically effective amount” refers to an amount that is sufficient to effect a therapeutically or prophylactically significant reduction in prion accumulation or prion activity when administered to a typical subject of the intended type. In aspects involving administration of an anti-prion oligoiucleotide to a subject, typically the oligonucleotide, formulation, or composition should be administered in a therapeutically effective amount.

As used herein in-connection with anti-prion oligonucleotides and formulations, and the like, in reference to a particular prion disease the term “targeted” indicates that the oligonucleotide is selected to inhibit development and/or aggregation of PrPsc, and/or development and/or progress of that particular prion disease. As used in connection with a particular tissue or cell type, the term indicates that the oligonucleotide, formulation, or delivery system is selected such that the oligonucleotide is preferentially present and/or preferentially exhibits an anti-prion effect in or proximal to the particular tissue or cell type.

As used in connection with the present oligos, the term “TG-rich” indicates that the sequence of the anti-prion oligonucleotide consists of at least 70 percent T and G nucleotides, or if so specified, at least 80, 90, or 95% T and G, or even 100%.

Selected Abbreviations

ON: Oligonucleotide
ODN: Oligodeoxynucleotide
PS: Phosphorothioate
TSE: Transmissible spongiform encephalopathies
PrPsc: Abnormal isoform prion protein
PrPc: Normal host encoded prion protein
CJD: Creutzfeldt-Jacob Disease
BSE: Bovine spongiform encephalopathy
CNS: Central Nervous System

Additional aspects and embodiments will be apparent from the following Detailed Description and from the claims.

### DETAILED DESCRIPTION OF THE PREFERRED EMBODIMENTS

I. General

The present invention is concerned with the identification and use of anti-prion ONs that act by a sequence independent mechanism, and includes the discovery that the anti-prion activity is greater for larger ONs that are 10 bases in length; typically 20 bases or more in length; and more preferably 40 and more bases in length (e.g., 20-60, 40-80, 60-100, 80-120.

As demonstrated by the results in Example 1, the anti-prion effect of random PS-ONs is not sequence specific or due to the action of an aptamer. Considering the volumes and concentrations of PS-ONs used in those tests, it is theoretically unlikely that a particular sequence is present at more than 1 copy in the mixture. This means than there can be no antisense or aptameric effect in these PS-ONs randomers. In all examples, should the anti-prion effect be caused by the sequence-specificity of the PS-ONs, such effect would thus have to be caused by only one molecule, a result that does not appear plausible. For example, for an ON randomer 40 bases in length, any particular sequence in the population would theoretically represent only ¼⁴⁰ or 8.27×10⁻²⁵ of the total fraction. Given that 1 mole=6.022×10²³ molecules, and the fact that our largest synthesis is currently done at the 15 micromole scale, all possible sequences will not be present and also, each sequence is present most probably as only one copy. Without limitation, a non-sequence dependent mode of action can be demonstrated by satisfying either Test 1 or Test 2 in Example 2.

Of course, one skilled in the art applying the teaching of the present invention could also use sequence specific ONs, but utilize the sequence independent activity discovered in the present invention. Accordingly, the present invention is not to be restricted to sequence independent ONs.

In the present invention, randomers (or other ONs) may inhibit prion diseases by several mechanisms, including but not limited to the following: inhibiting the conversion of PrPc to PrPsc, inhibiting the assembly of PrPsc, inhibiting the formation of amyloid plaques, inhibiting internalization of PrPc or PrPsc, rendering PrPsc sensitive to intra or extracellular proteases, preventing the precipitation of PrPsc and/or preventing the polymerization of PrPsc. While the preceding are suggested are potential mechanisms, the present invention is not limited thereby.

II. Anti-prion ONs

According to the conclusions discussed above and the data reported herein, ONs, e.g., ON randomers such as ODN random ers, have activity against the various types of prion disease.

Chemical Factors for Inhibition of Prion Activity

In Example 1, it is shown that PS modified ODN randomers exhibit potent anti-prion activity. This observation indicates that the anti-prion activity is involves the protein binding ability of the ON randomer.

One skilled in the art applying the teaching of the present invention can also use ONs with different chemical modifications. A modification of the ON, such as, but not limited to a PS modification, appears to be beneficial for anti-prion activity. This is most likely due to the effects of charge of ONs and/or to the requirement for stabilization of nucleic acids, e.g., DNA, both in the media and intracellularly, and/or the fact that thioated linkages promote protein binding. In addition, a specific chirality of each ihioated linkage (R versus P) may also be important for PS-ON randomer anti-prion activity.

Design of Non Sequence-specific ONs

It can also be advantageous to design or select anti-prion ONs demonstrating low (preferably the lowest possible) homology with the human (or other subject organism) genome. The goal is to obtain an ON that will show the lowest toxicity due to interactions with human or aniimal genome sequence(s) and mRNAs. The first step is to produce the desired length sequence of the ON, e.g., by aligning nucleotides A, C, G, T in a random fashion, manually or, more commonly, using a computer program. The second step is to compare the ON sequence with a library of human sequences such as GenBank and/or the Ensemble Human Genome Database. The sequence generation and comparison can be performed repetitively, if desired, to identify a sequence or sequences having a desired low homology level with the subject genome. It is desireable for the ON sequence to have the lowest homology possible with the entire genome or with mRNAs from the organism, while also minimizing self interaction. The last step is to test the ON in a prion assay using the suitable encapsulation to obtain anti-prion activity.

ONs Combining Non Sequence-specific Sequence with Antisense Sequence

In certain applications it can be desirable to couple a non-sequence specific ON sequence portion(s) with an antisense sequence portion(s) to increase the activity of the final ON. The non-sequence specific portion of the ONs is described in the present invention. The antisense portion is complementary to a MRNA of a gene involved in prion disease. One aim of this ON is to lower the expression of the PrP gene by combining a portion complementary to the mRNA of the Prp gene to the ON described herein.

ONs Combining Non-Sequence-specific Sequence with G-rich Sequence

In another approach, non-sequence specific sequence portion(s) is/are coupled with a G-rich motif ON portion(s) to improve the activity of the final ON. The non-specific portion of the ON is described in the present invention. The G-rich motif portion can, as non-limiting examples, include, CpQ, Gquartet, and/or CG that are described in the literature as stimulators of the immune system.

Non-Watson-Crick ONs

It can also be beneficial to use an ON composed of one or more types of non-Watson-Crick nucleotides/nucleosides. Such ONs can mimic PS-ONs and other modifications with some of the following characteristics similar to PS-ONs: a) the total charge; b) the space between the units; c) the length of the chain; d) a net dipole with accumulation of negative charge on one side; e) the ability to bind to proteins f) the ability to be encapsulate with delivery systems, h) an acceptable therapeutic index, i) an anti-prion activity. The ON has a preferred phosphorothioate backbone but is not limited to it. Examples of non-Watson-Crick nucleotides/nucleosides are described in Kool, 2002, Acc. Chem. Res. 35:936-943; and Takeshita et al., (1987) J. Biol. Chem. 262:10171-10179 where ONs containing synthetic abasic sites are described.

Linked ONs

In certain embodiments, ONs of the invention are modified in a number of ways without compromising their anti-prion activity. For example the ONs are linked or conjugated, at one or more of their nucleotide residues, to another moiety. Thus, modification of the oligonucleotides of the invention can involve chemically linking to the oligonucleotide one or more moieties or conjugates which enhance the activity, cellular distribution or cellular uptake, increase transfer across cellular membranes specifically or not, or protecting against degradation or excretion, or providing other advantageous characteristics. Such advantageous characteristics can, for example, include lower serum interaction, higher PrPsc interaction, the ability to be formulated for delivery, a detectable signal, improved pharmacokinetic properties, and lower toxicity: Such conjugate groups can be covalently bound to functional groups such as primary or secondary hydroxyl groups. For example, conjugate moieties can include a steroid molecule, a non-aromatic lipophilic molecule, a peptide, cholesterol, bis-cholesterol, an antibody, PEG, a protein, a water soluble vitamin, a lipid soluble vitamin, another ON, or any other molecule improving the activity and/or bioavailability of ONs.

In greater detail, exemplary conjugate groups of the invention can include intercalators, reporter molecules, polyamines, polyamides, polyethylene glycols, polyethers, SATE, t-butyl-SATE, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, fluorescent nucleobases, and dyes.

Groups that enhance the pharmacodynamic properties, in the context of this invention, include groups that improve oligomer cellular uptake and/or enhance oligomer resistance to degradation and/or protect against serum interaction. Groups that enhance the pharmacokinetic properties, in the context of this invention, include groups that improve oligomer uptake, distribution, metabolism or excretion. Exemplary conjugate groups are described in International Patent Application PCT/US92/09196, filed Oct. 23, 1992, which is incorporated herein by reference in its entirety.

Conjugate moieties can include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et at., EMBO J., 1991, 10, 1111-1118; Kabanov et al., FEBS Lett., 1990, 259, 327-330; Svinarchuk et at., Biochimie, 1993, 75,49-54), a phospholipid, e.g., di-hexadecyl-rac-glycerol or triethyl-ammonium 1,2-di-O-hexadecyl-rac-glycero-3-H-phosphonate (Manoharan et at., Tetrahedron Lett., 1995, 36, 3651-3654; Shea et al., Nucl Acids Res., 1990, 18, 3777-3783), a polyamine or a polyethylene glycol chain (Manoharan et at., Nucleosides & Nucleotides, 1995, 14, 969-973), or adamantane acetic acid (Manoharan et at., Tetrahedron Lett., 1995, 36, 3651-3654), a palmityl moiety (Mishra et at., Biochim. Biophys. Acta, 1995, 1264, 229-237), or an octadecylamine or hexylaminocarbonyl-oxycholesterol moiety (Crooke et al., J. Pharmacol Exp. Ther., 1996, 277, 923-937.

The present oligonucleotides may also be conjugated to active drug substances, for example without limitation, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

Exemplary U.S. patents that describe the preparation of exemplary oligonucleotide conjugates include, for example, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is incorporated by reference herein in its entirety.

Another approach is to prepare anti-prion ONs as lipophilic pro-oligonucleotides by modification with enzymatically cleavable charge neutralizing adducts subh as s-acetylthio-ethyl or s-pivasloylthio-ethyl (Vives et al., 1999, Nucl Acids Res 27: 4071-4076). Such modifications have been shown to increase the uptake of ONs into cells.

Oligonucleotide Modifications and Synthesis

As indicated above, modified oligonucleotides are useful in this invention. Such modified oligonucleotides include, for example, oligonucleotides containing modified backbones or non-natural intemucleoside linkages. Oligonucleotides having modified backbones include those that retain a phosphorus atom in the backbone and those that do not have a phosphorus atom in the backbone.

Such modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoakylphosphonates, thionoalkylphosphotriesters, selenophosphates, carboranyl phosphate and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity typically include a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Preparation of oligonucleotides with phosphorus-containing linkages as indicated above are described, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is incorporated by reference herein in its entirety.

Some exemplary modified oligonucleotide backbones that do not include a phosphorus atom have backbones that are formed by short chain alkyl or cycloalkyl intemucleoside linkages, mixed heteroatom and alkyl or cycloalkyl intemucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formnacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N,O,S and CH₂ component parts. Particularly advantageous are backbone linkages that include one or more charged moieties. Examples of U.S. patents describing the preparation of the preceding oligonucleotides include U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is incorporated by reference herein in its entirety.

Modified oligonucleotides may also contain one or more substituted sugar moieties. For example, such oligonucleotides can include one of the following 2′-modifications: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, or 2′-O—(O-carboran-1-yl)methyl. Particular examples are O[(CH₂)ₙO]ₘCH₃, O(CH₂)˜OCH₃, O(CH₂)ₙNH₂, O(CH₂)ₙCH₃, O(CH₂)ₙONH₂, and O(CH₂)ₙON [(CH₂)ₙCH₃)]₂, where n and m are from 1 to 10. Other exemplary oligonucleotides include one of the following 2′-modifications: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a reporter group, an intercalator, a group for improving the pharmacokinetic properties of an oligonucleotide, or a group for improving the pharmacodynamic properties of an oligonucleotide. Examples include 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group; 2′-dimethy-laminooxyethoxy, i.e., a O(CH₂)₂ON(CH₃)₂ group, also known as 2′-DMAOE; and 2′-dimethylaminoethoxyethoxy (also known as 2′-O-dimethylaminoethoxyethyl or 2′-DMAEOE), i.e., 2′-O—CH₂—O—CH₂—N(CH₂)₂.

Other modifications include Locked Nucleic Acids (LNAs) in which the 2′-hydroxyl group is linked to the 3′ or 4′ carbon atom of the sugar ring thereby forming a bicyclic sugar moiety. The linkage can be a methelyne (—CH₂—)˜group bridging the 2′ oxygen atom and the 4′ carbon atom wherein n is 1 or 2. LNAs and preparation thereof are described in WO 98/39352 and WO 99/14226, which are incorporated herein by reference in their entireties.

Other modifications include sulfur-nitrogen bridge modifications, such as locked nucleic acid as described in Orum et al. (2001) Curr. Opin. Mol. Ther. 3:239-243.

Other modifications include 2′-methoxy (2′-O—CH₃), 2′-methoxyethyl (2′O—CH₂-CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. Similar modifications may also be made at other positions on the oligonucleotide, particularly the 3′ position of the sugar on the 3′ terminal nucleotide or in 2′-5′ linked oligonucleotides and the 5′ position of the 5′ terminal nucleotide. Oligonucleotides may also have sugar mimetics such as cyclobutyl moieties in place of the pentofliranosyl sugar. Exemplary U.S. patents describing the preparation of such modified sugar structures include, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is incorporated by reference herein in its entirety.

Still other modifications include an ON concatemer consisting of multiple oligonucleotide sequences joined by a linker(s). The linker may, for example, consist of modified nucleotides or non-nucleotide units. In some embodiments, the linker provides flexibility to the ON concatemer. Use of such ON concatemers can provide a facile method to synthesize a final molecule, by joining smaller oligonucleotide building blocks to obtain the desired length. For example, a 12 carbon linker (C12 phosphoramidite) can be used to join two or more ON concatemers and provide length, stability, and flexibility.

As used herein, “unmodified” or “natural” bases (nucleobases) include the purine bases adenine (A) and guanine (G), and the pyrimidine bases thymine (T), cytosine (C) and uracil (U). Oligonucleotides may also include base modifications or substitutions. Modified bases include other synthetic and naturally-occurning bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Additional modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido [5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further nucleobases include those described in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applicattons, pages 289-302, Crooke, S. T. and Lebleu, B., ed., CRC Press, 1993.

Another modification includes phosphorodithioate linkages. Knowing that phosphorodithioate ODNs (PS2-ODNs) and PS-ODNs have a similar binding affinity to proteins (Tonkinson et al. (1994) Antisense Res. Dev. 4 :269-278)(Cheng et al. (1997) J. Mol. Recogn. 10:101-107) and knowing that a possible mechanism of action of ONs is binding to PrP, it could be desirable to include phosphorodithioate linkages on the anti-prion ONs described in this invention.

Another approach to modify ONs is to produce stereodefined or stereo-enriched ONs as described in Yu at al (2000) Bioorg. Med. Chem. 8:275-284 and in Inagawa et al. (2002) FEBS Lett. 25:48-52. ONs prepared by conventional methods consist of a mixture of diastereomers by virtue of the asymmetry around the phosphorus atom involved in the internucleotide linkage. This may affect the stability of the binding between ONs and PrP's. Previous data showed that protein binding is significantly stereo-dependent (Yu et al.). Thus, using stereodefined or stereo-enriched ONs could improve their protein binding properties and improve their anti-prion efficacy. In particular embodiments, the enrichment is at least 2-fold, 4-fold, 6-fold, 10-fold, 20-fold, 40-fold, 60-fold, 80-fold, 100-fold or even more.

The incorporation of modifications such as those described above can be utilized in many different incorporation patterns and levels. That is, a particular modification need not be included at each nucleotide or linkage in an oligonucleotide, and different modifications can be utilized in combination in a single bligonucleotide, or even in a single nucleotide.

Oligonucleotide Synthesis

The present oligonucleotides can by synthesized using methods known in the art. For example, unsubstituted and substituted phosphodiester (P═O) oligonucleotides can be synthesized on an automated DNA synthesizer (e.g., Applied Biosystems model 380B) using standard phosphorarnidite chemistry with oxidation by iodine. Phosphorothioates P═S) can be synthesized as for the phosphodiester oligonucleotides except the standard oxidation bottle can be replaced by 0.2 M solution of 311-1,2-benzodithiole-3-one 1,1-dioxide in acetonitrile for the step-wise thioation of the phosphite linkages. The thioation wait step can be increased to 68 sec, followed by the capping step. After cleavage from the CPG column and deblocling in concentrated ammonium hydroxide at 55° C. (18 h), the oligonucleotides can be purified by precipitating twice with 2.5 volumes of ethanol from a 0.5 M NaCl solution.

Phosphinate oligonucleotides can be prepared as described in U.S. Pat. No. 5,508,270; alkyl phosphonate oligonucleotides can be prepared as described in U.S. Pat. No. 4,469,863; 3′-Deoxy-3′-methylene phosphonate oligonucleotides can be prepared as described in U.S. Pat. Nos. 5,610,289 and 5,625,050; phosphoramidite oligonucleotides can be prepared as described in U.S. Pat. No. 5,256,775 and U.S. Pat. No. 5,366,878; alkylphosphonothioate oligonucleotides can be prepared as described in published PCT applications PCT/US94/00902 and PCT/US93/06976 (published as WO 94/17093 and WO 94/02499, respectively); 3′-Deoxy-3′-amino phosphoramidate oligonucleotides can be prepared as described in U.S. Pat. No. 5,476,925; Phosphotriester oligonucleotides can be prepared as described in U.S. Pat. No. 5,023,243; borano phosphate oligonucleotides can be prepared as described in U.S. Pat. Nos. 5,130,302 and 5,177,198; methylenemethylimino linked oligonucleotides, also identified as MMI linked oligonucleotides, methylenedimethyl-hydrazo linked oligonucleotides, also identified as MDII linked oligonucleotides, and methylenecarbonylamino linked oligonucleotides, also identified as amide-3 linked oligonucleotides, and methyleneaminocarbonyl linked oligo-nucleotides, also identified as amide-4 linked oligonucleo-sides, as well as mixed backbone compounds having, for instance, alternating MMI and P═O or P═S linkages can be prepared as described in U.S. Pat. Nos. 5,378,825, 5,386,023, 5,489,677, 5,602,240 and 5,610,289; formacetal and thioformacetal linked oligonucleotides can be prepared as described in U.S. Pat. Nos. 5,264,562 and 5,264,564; and ethylene oxide linked oligonucleotides can be prepared as described in U.S. Pat. No. 5,223,618. Each of the cited patents and patent applications is incorporated by reference herein in its entirety.

Concurrent Use of Anti-prion Polymers with Inhibition of PrP Expression

The present oligonucleotides, e.g., ONs, can also be used concurrently with an agent that inhibits expression of PrPc. As known in the art, a variety of different types of inhibitors can be used, including, for example, ribozymes or other catalytic nucleic acid molecules, antisense, triple helix, and RNAi (e.g., using siRNA or shRNA which can be prepared synthetically or can be-expressed intracellularly). RNAi, and specifically siRNA is described in numerous references, including for example, Fire et al., U.S. Pat. No. 6,506,559, issued Jan. 14, 2003; Graham et al., U.S. Pat. No. 6,573,099, issued Jun. 3, 2003; Zemicka-Goetz et al.; US publ. 20030027783, published Feb. 6, 2003; application Ser. 10/150,426, filed May 5, 2002; Tuschl et al. (1) published appl. 20020086356, application Ser. No. 09/821,832, filed Mar. 30, 2001, each of which is incorporated herein by reference in its entirety.

In such a concurrent approach, the anti-prion oligonucleotides and the PrPc expression inhibitor can be delivered together or separately, which can be by the same or different delivery routes and/or methods.

Polymers with Prion Inhibition Properties

Another approach is to use a polymer mimicking the activity of ONs described in the present invention and encapsulate it with suitable delivery system in order to provide inhibition of prion activity. As described in the literature, several anionic polymers were shown to bind to proteins. These polymers belong to several classes: (1) sulfate esters of polysaccharides (dextrin and dextran sulfates, cellulose sulfate); (2) polymers containing sulfonated benzene or naphthalene rings and naphthalene sulfonate polymers; (3) polycarboxylates (acrylic acid polymers); and acetyl phthaloyl cellulose (Neurath et al. (2002) BMC Infect Dis 2:27); and (4) abasic oligonucleotides (Takeshita et al., 1987, J. Biol. Chem. 262:10171-10179). Other examples of non-nucleotide protein binding polymers are described in the literature. The polymers described herein mimic ONs described in this invention and have the following characteristics similar to ONs: a) the length of the chain; b) a net dipole with accumulation of negative charge on one side; c) the ability to bind to proteins; d) the ability to be encapsulated by a delivery system, e) an acceptable therapeutic index, f) an anti-prion activity. In order to mimic the effect of an ON, the anti-prion polymer may preferably be a polyanion displaying similar space between its units as compared to a PS-ON. It may also have the ability to penetrate cells with a delivery system.

It may also be to possible to modify polymers which normally do not have a anionic character, for instance polyethylene imine, by the incorporation of sulfuir and or oxygen and or other modifications which result in the conversion of the resultant polymer from a neutral or cationic polymer into a polyanion. This technique could be applied to any and all suitable polymers. Since we have evidence that the polyanionic nature of PS-ON randomers forms the basis of their anti-prion activity, we believe that any particular molecule with a polyanionic character (e.g., carbohydrate polymers or oligonucleotides) will have anfi-prion activity.

Anti-prion Activity of Double-stranded ONs

According to our results described herein, an approach is to use double stranded ONs as effective anti-prion agent with or without an encapsulating agent to deliver it. Preferentially such ONs have a phosphorothioate backbone but may also have other/additional modifications which improve their pharmacokinetic behaviour and/or anti-prion activity and/or stability as described herein for single stranded ONs.

III. Treatment of Blood and Blood Products.

Conventional antiseptic compositions and antiseptic methodologies are generally insufficient for inactivating infectious proteins such as prions. Although prions can be inactivated by relatively high temperatures over very long periods of time, the temperature ranges and time periods generally used to kill bacteria and inactivate the viruses are insufficient to inactivate prions. Temperature treatment may also alter or destroy required characteristics of blood and blood products.

Thus, the present invention also concerns the use of the ONs and polyanions described herein in methods to treat blood and blood products prior to transfer to a human or animal. Application of the ON or polyanion of the invention can render prions non-infectious and/or prevent prion formation and/or aid in the denaturation of prions from blood and blood products. An important aspect of the invention is that the active component be able to eliminate infectivity or denature an infectious protein such as PrP under relatively mild conditions in order to conserve the desired blood characteristics. The protocol for treatment includes a step where the ON or polyanion is put in contact with the blood or blood product for a determined amount of time. This treatment may also be done on whole blood prior to blood product processing steps or during any processing steps. ONs may also be used in combination with other physical or chemical blood treatments such as temperature, radiation, and aseptic compositions.

Similarly, ONs or polyanions as described herein can be used to treat tissue or organs to be transplanted to humans or animals.

Likewise, immobilizd ONs or polyanions can be used in a method of for removal of PrP proteins from blood or blood products. The ON or polyanion immobilized on a solid phase support or membrane common in a variety of purification procedures can be used to remove prions from a biological material. A number of methods for use in the present invention are summarized as follows.

Methods of Purification

Another method that may be used to remove prions from a biological sample involves filtration, through a membrane. The membrane may have the ON or polyanion conjugated directly to the membrane or alternatively, the ON or polyanion may be compartmentalized in an area behind the membrane, which is inaccessible to the larger components of the biological materials, e.g. blood cells. In the latter example, the ON or polyanion can be bound to an insoluble matrix behind the membrane. Suitable materials for the membrane include without limitation regenerated cellulose, cellulose acetate, non-woven acrylic copolymer, polysiilphone, polyether sulphone, polyacrylonitrile, polyamide and the like. The ON or polyanion is immobilized in the pores and/or on the surface of the side of the membrane that faces away from the biological fluid.

Alternatively, the ON may be bound to a solid matrix and used on an affinity chromatography column. A number of matrices may be employed in the preparation of columns. Such matrices can include without limitation beads, and more preferably spherical beads, which serve as a support surface for the complexing agent of the invention. Suggested materials for the matrices include without limitation agarose, cross-linked dextran, polyhydroxyl ethyl methacrylate, polyacrylamide, polyurethane, cellulose, cellulose acetate and derivatives or combinations thereof. Those skilled in the use of such materials are familiar with techniques for binding or linking oligonucleotides and polymers as described herein to such matrices, and such techniques can be used with the present invention.

### REFERENCES CORRESPONDING TO ABBREVIATED CITATIONS IN TEXT

Adjou K T, Simoneau S, Sales N, Lamoury F, Dormont D, Papy-Garcia D, Barritault D, Deslys J P, Lasmezas C I. A novel generation of heparan sulfate mimetics for the treatment of prion diseases. J Gen Virol. 2003 September;84(Pt 9):2595-603.
Banks W A, Farr S A, Buft W, Kumar V B, Franko M W, Morley J E. Delivery across the blood-brain barrier of antisense directed against amyloid beta: reversal of learning and memory deficits in mice overexpressing amyloid precursor protein. J Pharmacol Exp Ther. 2001 June;297(3):1113-21.
Barret A, Tagliavini F, Forloni G, Bate C, Salmona M, Colombo L, De Luigi A, Limido L, Suardi S, Rossi G, Auvre F, Adjou K T, Sales N, Williams A, Lasmezas C, Deslys J P. Evaluation of quinacrine treatment for prion diseases. J Virol. 2003 August;77(15):8462-9.
Bate C, Salmona M, Diomede L, Williams A. Squalestatin cures prion-infected neurons and protects against prion neurotoxicity. J Biol Chem. 2004 Apr. 9;279(15):14983-90.
Brigger I, Morizet J, Aubert G, Chacun H, Terrier-Lacombe M J, Couvreur P, Vassal Poly(ethylene glycol)-coated hexadecylcyanoacrylate nanospheres display a combined effect for brain tumor targeting. J Pharmacol Exp Ther. 2002 December;303(3):928-36.
Caughey B, Raymond G J. Sulfated polyanion inhibition of scrapie-associated PrP accumulation in cultured cells. J Virol. 1993 February;67(2):643-50.
Doh-ura K, Ishikawa K, Murakami-Kubo I, Sasaki K, Mohri S, Race R, Iwaki T. Treatment of transmissible spongiform encephalopathy by intraventricular drug infusion in animal models. J Virol. 2004 May;78(10):4999-5006.
Grigoriev V B, Adjou K T, Sales N, Simoneau S, Deslys J P, Seman M, Dormont D, Fournier J G. Effects of the polyene antibiotic derivative MS-8209 on the astrocyte lysosomal system of scrapie-infected hamsters. J Mol Neurosci. 2002 June;18(3):271-81.
Huwyler J, Wu D, Pardridge W M. Brain drug delivery of small molecules using immunoliposomes Proc Natl Acad Sci U S A. 1996 Nov. 26;93(24):14164-9.
Kocisko D A, Baron G S, Rubenstein R, Chen J, Kuizon S, Caughey B. New inhibitors of scrapie-associated prion protein formation in a library of 2000 drugs and natural products. J Virol. 2003 October;77(19):10288-94.
Koster T, Singh K, Zimmermann M, Gruys E. Emerging therapeutic agents for transmissible spongiform encephalopathies: a review. J Vet Pharmacol Ther. 2003 October;26(5):315-26.
Nakajima M, Yamada T, Kusuhara T, Furukawa H, Takahashi M, Yamauchi A, Kataoka Y. Results of quinacrine administration to patients with Creutzfeldt-Jakob disease. Dement Geriatr Cogn Disord. 2004;17(3):158-63.
Omori N, Maruyama K, Jin G, Li F, Wang S J, Hamakawa Y, Sato K, Nagano I, Shoji M, Abe K. Targeting of post-ischemic cerebral endothelium in rat by liposomes bearing polyethylene glycol-coupled transferrin. Neurol Res. 2003 April;25(3):275-9.
Poli G, Ponti W, Carcassola G, Ceciliani F, Colombo L, Dall'Ara P, Gervasoni M, Giannino M L, Martino P A, Pollera C, Villa S, Salmona M. In vitro evaluation of the anti-prionic activity of newly synthesized congo red derivatives. Arzneimittelforschung. 2003;53(12):875-88.
Priola S A, Raines A, Caughey W S. Porphyrin and phthalocyanine antiscrapie compounds. Science. 2000 Feb. 25;287(5457):1503-6.
Schmidt J, Metselaar J M, Wauben M H, Toyka K V, Storm G, Gold R. Drug targeting by long-circulating liposomal glucocorticosteroids increases therapeutic efficacy in a model of multiple sclerosis. Brain. 2003 August;126(Pt 8):1895-904.
Shyng S L, Lehmann S, Moulder K L, Harris D A. Sulfated glycans stimulate endocytosis of the cellular isoform of the prion protein, PrPC, in cultured cells. J Biol Chem. 1995 Dec. 15;270(50):30221-9.
Supattapone S, Wille H, Uyechi L, Safar J, Tremblay P, Szoka F C, Cohen F E, Prusiner S B, Scott M R. Branched polyamines cure prion-infected neuroblastoma cells. J Virol. 2001 April;75(7):3453-61.
Vinogradov S V, Batrakova E V, Kabanov A V. Nanogels for oligonucleotide delivery to the brain. Bioconjug Chem. 2004 January-February;15(1):50-60.
Zhang X, Xie J, Li S, Wang X, Hou X. The study on brain targeting of the amphotericin B liposomes. J Drug Target. 2003 February;11(2):1 17-22.
Zhang Y, Schlachetzki F, Pardridge W M. Global non-viral gene transfer to the primate brain following intravenous administration. Mol Ther. 2003 January;7(1): 11-8.
   IV. Pharmaceutical Compositions and Delivery
   Pharmaceutical Compositions

The ONs of the invention may be in the form of a therapeutic composition or formulation useful for treating (or prophylaxis of) a prion disease or diseases, which can be approved by a regulatory agency for use in humans or in non-human animals, and/or against a particular prion disease. These ONs may be used as part of a pharmaceutical composition when combined with a physiologically and/or pharmaceutically acceptable carrier. The characteristics of the carrier may depend on the route of administration. The pharmaceutical composition of the invention may also contain other active factors and/or agents which enhance activity.

Administration of the ONs of the invention used in the pharmaceutical composition or formulation or to practice the method of treating an animal can be carried out in a variety of conventional ways, such as intraocular, oral ingestion, enterally, inhalation (using a wet or dry aerosol), or cutaneous, subcutaneous, intramuscular, intraperitoneal, intrathecal, intratracheal, intracerebral, intracranial, intraventricular or intravenous injection.

The pharmaceutical composition or oligonucleotide formulation of the invention may further contain other anti-prion agents, e.g., one or more PrPc expression inhibitors.

The pharmaceutical composition or oligonucleotide formulation of the invention may further contain a polymer, such as, without restriction, polyanionic agents, sulfated polysaccharides, heparin, dextran sulfate, pentosan polysulfate, polyvinylalcool sulfate, acemannan, polyhydroxycarboxylates, cellulose sulfate, polymers containing sulfonated benzene or naphthalene rings and naphthalene sulfonate polymer, acetyl phthaloyl cellulose, poly-L-lysine, sodium caprate, cationic amphiphiles, cholic acid.

Oligonucleotide Formulations and Pharmaceutical Compositions

The present oligonucleotides can be prepared in an oligonucleotide formulation or pharmaceutical composition. Thus, the present oligonucleotides may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Exemplary United States patents that describe the preparation of such uptake, distribution and/or absorption assisting formulations include, for example, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is incorporated herein by reference in its entirety.

The oligonucleotides, formulations, and compositions of the invention include any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular embodiments, prodrug versions of the present oligonucleotides are prepared as SATE [(S-acetyl-2-thioethyl) phosphate] derivatives according to the methods disclosed in Gosselin et al., WO 93/24510 and in Imbach et al., WO 94/26764 and U.S. Pat. No. 5,770,713, which are hereby incorporated by reference in their entireties.

The term “pharmaceutically acceptable salts” refers to physiologically and pharmaceutically acceptable salts of the present compounds: i.e., salts that retain the desired biological activity of the parent compound and do not impart undesired toxicological effects thereto. Many such pharmaceutically acceptable salts are known and can be used in the present invention.

For oligonucleotides, useful examples of pharmaceutically acceptable salts include but are not limited to salts formed with cations such as sodium, potassium, ammonium, magnesium, calcium, polyamines such as spermine and spermidine, etc.; acid addition salts formed with inorganic acids, for example hydrochloric acid, hydrobromic acid, sulfuric acid, phosphoric acid, nitric acid and the like; salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and salts formed from elemental anions such as chlorine, bromine, and iodine.

The present invention also includes pharmaceutical compositions and formulations which contain the anti-prion oligonucleotides of the invention. Such pharmaceutical compositions may be administered in a number of ways depending upon whether local or systemic treatment is desired and upon the area to be treated. For example, administration may be topical (including ophthalmic and to mucous membranes including vaginal and rectal delivery); pulmonary, e.g., by inhalation or insufflation of powders or aerosols, including by nebulizer; intratracheal; intranasal; epidermal and transdermal; oral; or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, laurie acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Exemplary surfactants include fatty acids and/or esters or salts thereof, bile acids and/or salts thereof. Exemplary bile acids/salts include chenodeoxycholic acid (CDCA) and ursodeoxychenedeoxycholic acid (IDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Exemplary fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further exemplary penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include poly-amino acids; polyimines; polyacrytates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses, and starches. Particularly advantageous complexing agents include chitosan, N-trimethytchitosan, poly-L-lysine, polyhistidine, polyorithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylatc), poly(isobutylcyanoacrylate), poly(isohexylcynaoacrylate), DEAE-methacrylate, DEAE-hexylacrylate, DEAE-acrylamide, DEAE-albumin and DEAB-dextran, polymethylacrylate, polyhexylacrylate, poly(D,L-lactic acid), poly(DL-lactic-co-glycolic acid (PLGA), alginate, and polyethyleneglycol (PEG).

Compositions and formulations for parenteral, intracranial, intracerebral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaking the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

Emulsions

The formulations and compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (lids.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et at., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: non-ionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include-polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong inter-facial films around the dispersed-phase droplets and by increasing the viscosity of the external phase.

Since emulsions often contain a number of ingredients such as carbohydrates, proteins, sterols and phosphatides that may readily support the growth of mnicrobes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailabiity standpoint. (Rosoff, in Pharmaceutical Dosage Forms, Lieberman, Rieger and Banker (Eds.), 1988, Marcel Dekker, Inc., New York, N.Y., volume 1, p. 245; ldson, in Pharmaceutical Dosage Forms, Lieberman, Rieger and Banker (ds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically micro-emulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington 's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drag, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils-and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschet, Met/i. Find. Exp. Clin. PharmacoL, 1993, 13, 205). Micro-emulsions afford advantages of improved drug solubilization, protection of drug from.enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et at., Pharmaceutical Research, 1994, 11, 1385; Ho et al., J. Pharm. Set, 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucteotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92).

Liposomes

There are many organized surfactant structures besides microemulsions that have been studied and used for the formulation of drugs. These include monolayers, micelles, bilayers and vesicles. Vesicles offer specificity and extended duration of action for drug delivery. Thus, as used herein, the term “liposome” refers to a vesicle composed of amphiphilic lipids arranged in a spherical bilayer or bilayers, i.e., liposomes are unilamellar or multilamellar vesicles which have a membrane formed from a lipophilic material and an aqueous interior. The aqueous portion typically contains the composition to be delivered. In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores. Additional factors for liposomes include the lipid surface charge, and the aqueous volume of the liposomes.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245).

For topical administration; there is evidence that liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin. Compounds including analgesics, antibodies, hormones and high-molecular weight DNAs have been administered to the skin, generally resulting in targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et at., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. The DNA is thus entrapped in the aqueous interior of these liposomes. pH-sensitive liposomes have been used, for example, to deliver DNA encoding the thymidine kinase gene to cell monolayers in culture (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et at., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasone™ I (glyceryl dilaurate/cholesterolpolyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et at. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome include one or more glycolipids, such as monosialoganglioside G_{M1}, or is derivatized with one or more hydrophilic polymers, such as a polyethylene glycol (PEG) moiety. Without being bound by any particular theory, it is believed that for sterically stabilized liposomes containing gangliosides, sphingomyelin, or PEG-derivatized lipids, the increase in circulation half-life of these sterically stabilized liposomes is due to a reduced uptake into cells of the reticuloendothelial system (RES) (Allen et at., FEBS Lett., 1987, 223, 42; Wu et al., Cancer Research, 1993, 53, 3765).

Various liposomes that include one or more glycolipids have been reported in Papahadjopoulos et al., Ann. N.Y. Acad. Sci., 1987, 507, 64 (monosiatoganglioside G_{Ml}, galactocerebroside sulfate and phosphatidylinositol); Gabizon et at., Proc. Natl. Acad. Sci. USA., 1988, 85, 6949,;Allen et al., US. Pat. No. 4,837,028 and International Application Publication WO 88/04924 (sphingomyelin and the ganglioside G_{M1} or a galactocerebroside sulfate ester); Webb et al., U.S. Pat. No. 5,543,152 (sphingomyelin); Lim et al., WO 97/13499 (1,2-sn-dimyrstoylphosphatidylcholine).

Liposomes that include lipids derivatized with one or more hydrophilic polymers, and methods of preparation are described, for example, in Sunamoto et al., Bull. Chem. Soc. Jpn., 1980, 53, 2778 (a nonionic detergent, 2C₁₂15G, that contains a PEG moiety); Illum et al., FEBS Lett., 1984, 167, 79 (hydrophilic coating of polystyrene particles with polymeric glycols); Sears, U.S. Pat. Nos. 4,426,330 and 4,534,899 (synthetic phospholipids modified by the attachment of carboxylic groups of polyalkylene glycols (e.g., PEG)); Klibanov et al., FEBS Lett., 1990, 268, 235 (phosphatidylethanolamine (PE) derivatized with PEG or PEG stearate); Blume et al., Biochimica et Biophysica Acta, 1990, 1029, 91 (PEG-derivatized phospholipids, e.g., DSPE-PEG, formed from the combination of distearoylphosphatidylethanolamine (DSPE) and PEG); Fisher, European Patent No. EP 0 445 131 B 1 and WO 90/04384 (covalently bound PEG moieties on liposome external surface); Woodle et al., U.S. Pat. Nos. 5,013,556 and 5,356,633, and Martin et al., U.S. Pat. No. 5,213,804 and European Patent No. EP 0 496 813 B1 (liposome compositions containing 1-20 mole percent of PE derivatized with PEG); Martin et al., WO 91/05545 and U.S. Pat. No. 5,225,212 and in Zalipsky et al., WO 94/20073 (liposomes containing a number of other lipid-polymer conjugates); Choi et al., WO 96/10391 (liposomes that include PEG-modified ceramide lipids); Miyazaki et al., U.S. Pat. No. 5,540,935, and Tagawa et al., U.S. Pat. No. 5,556,948 (PEG-containing liposomes that can be further derivatized with functional moieties on their surfaces).

Liposomes that include nucleic acids have been described, for example, in Thierry et al., WO 96/40062 (methods for encapsulating high molecular weight nucleic acids in liposomes); Tagawa et al., U.S. Pat. No. 5,264,221 (protein-bonded liposomes containing RNA); Rahman et al., U.S. Pat. No. 5,665,710 (methods of encapsulating oligodeoxynucleotides in liposomes); Love et al., WO 97/04787 (liposomes that include antisense oligonucleotides).

Another type of liposome, transfersomes are highly deformable lipid aggregates which are attractive for drug delivery vehicles. (Cevc et al., 1998, Biochim Biophys Acta. 1368(2):201-15.) Transfersomes maybe described as lipid droplets which are so highly deformable that they can penetrate through pores which are smaller than the droplet. Transfersomes are adaptable to the environment in which they are used, for example, they are shape adaptive, self-repairing, frequently reach their targets without fragmenting, and often self-loading. Transfersomes can be made, for example, by adding surface edge-activators, usually surfactants, to a standard liposomal composition.

Surfactants

Surfactants are widely used in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants are widely used in pharmaceutical and cosmetic products and are usable over a wide range of pH values, and with typical HLB values from 2 to about 18 depending on structure. Nonionic surfactants include nonionic esters such as ethylene glycol esters, propylene glycol esters, glyceryl esters, polyglyceryl esters, sorbitan esters, sucrose esters, and ethoxylated esters; and nonionic alkanolamides and ethers such as fatty alcohol ethoxylates, propoxylated alcohols, and ethoxylated/propoxylated block polymers are also included in this class. The polyoxyethylene surfactants are the most commonly used members of the nonionic surfactant class.

Surfactant molecules that carry a negative charge when dissolved or dispersed in water are classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isothionates, acyl laurates and sulfosuccinates, and phosphates. The alkyl sulfates and soaps are the most conmnonly used anionic surfactants.

Surfactant molecules that carry a positive charge when dissolved or dispersed in water are classified as cationic. Cationic surfactants include quaternary ammonium salts and ethoxylated amines, with the quaternary ammonium salts used most often.

Surfactant molecules that can carry either a positive or negative charge are classified as amphoteric. Amphoteric surfactants include acrylic acid derivatives, substituted alkylamides, N-alkylbetaines and phosphatides.

The use of surfactants in drug products, formulations and in emulsions has been reviewed in Rieger, in Pharmaceutical Dosage Forms, Marcel Dekker, Inc., New York, N.Y., 1988, p. 285).

Penetration Enhancers

In some embodiments, penetration enhancers are used in or with a composition to increase the delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Exemplary penetration enhancers may be classified as belonging to one of five broad categories, i.e., surfactants, fatty acids, bile salts, chelating agents, and non-chelating nonsurfactants (Lee et al., Critical Reviews in Therapeutic Drug Carrier Systems, 1991, p.92). Each of these classes of penetration enhancers is described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. These penetration enhancers include, for example, sodium lauryl sulfate, polyoxyethylene-9-lauryl ether and polyoxyethylene-20-cetyl ether) (Lee et at., Critical Reviews in Therapeutic Drug Carrier Systems, 1991, p.92); and perfluorochemical emulsions, such as FC43. Takahashi et al., J. Pharm. Pharmacol., 1988, 40, 252), each of which is incorporated herein by reference in its entirety.

Fatty acids: Various fatty acids and their derivatives which act as penetration enhancers include, for example, oleic acid, lauric acid, capric acid (n-decanoic acid), myristic acid, paimitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and diglycerides thereof (i.e., oleate, laurate, caprate, myristate, palmitate, stearate, linoleate, etc.) (Lee et al., Critical Reviews in Therapeutic Drug Carrier Systems, 1991, p.92,; Muranishi, Critical Reviews in Therapeutic Drug Carrier Systems, 1990, 7, 1-33; El Hariri et al., J. Pharm. Pharmacol., 1992, 44, 651-654), each of which is incorporated herein by reference in its entirety.

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto ct al., J. Pharm. Exp. Ther., 1992, 263, 25; Yamashita et al., J. Pharm:. Sci., 1990, 79, 579-583).

Chelating Agents: In the present context, chelating agents can be regarded as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Without limitation, chelating agents include disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds are compounds that do not demonstrate significant chelating agent or surfactant activity, but still enhance absorption of oligonucleotides through the alimentary mucosa (Muranishi, Critical Reviews in Therapeutic Drug Carrier Systems, 1990, 7, 1-33). Examples of such penetration enhancers include unsaturated cyclic ureas, 1-alkyl- and 1-alkenylazacyclo-alkanone derivatives (Lee et al., Critical Reviews in Therapeutic Drug Carrier Systems, 1991, page 92); and nonsteroidal anti-inflammatory agents such as diclofenac sodium, indomethacin and phenylbutazone (Yamashita et al, J. Pharm. Pharmacol., 1987, 39, 621-626).

Agents that enhance uptake of oligonucleotides at the cellular level may also be added to the pharmaceutical and other compositions and formulations of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, often with an excess of the latter substance, can result in a substantial reduction of the amount of nucleic acid recovered in the liver, kidney or other extracirculatory reservoirs. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2-disulfonic acid (Miyao et al., Antisense Res. Dev., 1995,5, 115-121; Takakura et al., Antisense & Nucl Acid Drug Dev., 1996, 6, 177-183), each of which is incorporated herein by reference in its entirety.

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal, and is typically liquid or solid. A pharmaceutical carrier is generally selected to provide for the desired bulk, consistency, etc., when combined with a nucleic acid and the other components of a given pharmaceutical composition, in view of the intended administration mode. Typical pharmaceutical carriers include, buit are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycotate, etc.); and wetting agents (e.g., sodium lauryl sulphate, etc.).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Other Pharmaceutical Composition Components

The present compositions may additionally contain other components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran, and/or stabilizers.

Certain embodiments of the invention provide pharmaceutical compositions containing (a) one or more anti-prion oligonucleotides and (b) one or more other anti-prion agents which function by a different mechanism, e.g., PrPc expression inhibitors. Two or more combined compounds may be used together or sequentially.

CNS and Other Tissue Delivery

All prion-related diseases are characterized by neurological dysfuntion. This is due to the preferential accumulation of converted prion proteins in CNS neurons. Prion-mediated plaque formation in these neurons leads to altered neuronal function which is the pathology behind neurological impairment.

For any therapy against prion-related diseases to be effective, it must be easily delivered to the brain, the major site of prion accumulation. There is some evidence to indicate an active transport of ONs across the blood brain barrier (Banks et al., 2001), but naked ONs in general are not efficiently transported across the blood brain barrier, so that intrathecal, intraventricular, intracerebral, or intracranial injection can be effective routes for delivering an ON therapeutic. Since these routes of administration require surgical intervention, they are not preferable and are not convenient for multiple dose administration. However, there are several technologies which can be used to either limit the administration to a single dose or to allow ONs to more efficiently cross the blood brain barrier, thus opening up many other, preferable routes of administration (e.g., intravenous, subcutaneous, transdermal, inhalation).

Reservoirs of ONs (e.g. ALZET Osmotic Pumps, DURECT Corporation) can be used intracranially to deliver ONs to the brain over long periods. However the majority of technologies successfully employed to increase the delivery of ONs across the blood brain barrier involves the use of cationic liposomes or polycationic polymers which are known to effectively encapsulate ONs. These technologies include but are not limited to: pegylated polyethyleneimine nanogels (Vinogradov et al., 2004), the use of pegylated liposomes conjugated to antibodies directed against the insulin receptor (Zhang et al., 2003) or the transferrin receptor (Huwyler et al., 1996), direct conjugation of pegylated liposomes to transferrin (Omori et al.,2003), pegylated hexadecylcyanoacrylate nanospheres Brigger et al., 2002) or vasoactive peptide conjugated liposomes or pegylated liposomes (i.e. RMP-7; Zhang et al., 2003).

Since the PS-ON randomers described herein are compatible with all these delivery technologies or modifications, those technologies can be used to deliver PS-ON randomers across the blood brain barrier.

While the effects of PrPsc significantly relate to development of amyloid plaques in the CNS, it is advantageous to provide anti-prion activity to other tissues. Thus, additional delivery methods as described herein are also useful.

Thus, use of a delivery system can significantly increase the anti-prion potency of ON randomers. Additionally, they will serve to protect these compounds from serum interactions, reducing side effects and maximizing tissue and cellular distribution.

Although PS-ONs are more resistant to endogenous nucleases than natural phosphodiesters, they are not completely stable and are slowly degraded in blood and tissues. A limitation in the clinical application of PS oligonucleotide drugs is their propensity to activate complement on i.v. administration. In general, liposomes and other delivery systems enhance the therapeutic index of drugs, including ONs, by reducing drug toxicity, increasing residency time in the plasma, and delivering more active drug to tissue by extravasation of the carriers through hyperpermeable vasculature. Moreover in the case of PS-ON, lipid encapsulation prevents the interaction with potential protein-binding sites while in circulation (Klimuk et al. (2000) J Pharmacol Exp Ther 292:480-488).

According to our results, an advantageous approach is to use a delivery system such as, but without restriction, lipophilic molecules, polar lipids, liposomes, monolayers, bilayers, vesicles, programmable fusogenic vesicles, micelles, cyclodextrins, PEG, iontophoresis, powder injection, and nanoparticles (such as PIBCA, PIHCA, PHCA, gelatine, PEG-PLA) for the delivery of ONs described herein. The purpose of using such delivery systems are to, among other things, lower the toxicity of the active compound in animals and humans, increase cellular delivery, lower the IC50, increase the duration of action from the standpoint of drug delivery and protect the oligonucleotides from non-specific binding with serum proteins.

It is known in the art that one of the main therapeutic factors for phosphorothioate antisense oligonucleotides is their side effects due mainly to this increased interaction with proteins (specifically with serum proteins) as described by Kandimalla and co-workers (Kandimalla et al. (1998) Bioorg. Med. Chem. Lett. 8:2103-2108). Our data suggests substantial benefits by a suitable delivery system capable of delivering anti-prion ONs into the cell while preventing their interaction with serum proteins.

Another approach is to accomplish cell specific delivery by associating the delivery system with a molecule(s) that will increase affinity with specific cells, such molecules being without restriction antibodies, receptor ligands, vitamins, hormones and peptides.

### EXAMPLE 1

### Demonstration of Potent, Size-dependent PS-ODN Randomer Anti-prion Activity

The anti-PrP activity of PS-ODN randomers (prepared as single-stranded randomers) was tested in a tissue culture model of PrP conversion. Three PS-ODN randomers were used: REP 2003 (10 mer), REP 2004 (20 mer), and REP 2006 (40 mer).

Approximately 20,000 RML or 22L scrapie-infected mouse neuroblastoma cells were added to each well of a 96 well plate in 100 μL of medium prior to the addition of test compounds. 22L-infected cells were developed by re-infection of RML-infected mouse neuroblastoma cells cured by 7 passages in medium containing 1 μg/mL pentosan polysulfate. The cured cells were re-infected by incubation with PrPsc purified from mouse brains infected with 22L-strain of scrapie. The neuroblastoma cells reinfected with 22L scrapie have stably expressed PrPsc for over 70 passages. The cells were allowed to settle for 4 hours before test compounds were added.

PS-ODN randomers were diluted into PBS prior to being introduced to the cell medium. 5 μL of solutions were added to the cell medium. After PS-ODN randomers were added, the cells were incubated for 5 days at 37° C. in 5% CO₂ before being lysed.

Prior to cell lysis, the cells were inspected by light microscopy for toxicity, bacterial contamination, and density compared to controls. After removal of the cell media, 50 μL of lysis buffer was added to each well. Lysis buffer was composed of 0.5% (w/v) Triton X-100, 0.5% (w/v) sodium deoxycholate, 5 mM tris-HCl, pH 7.4 at 4° C., 5 mM EDTA, and 150 mM NaCl. Five minutes after adding lysis buffer, 25 μL of 0.1 mg/mL PK (Calbiochem) in TBS was added to each well and incubated at 37° C. for 50 minutes. 225 μL of 1 mM Pefabloc (Boehringer Mannheim) was then added to each well to inhibit PK activity. 250 μL of 1 mM Pefabloc was added to samples that were not PK-treated.

To detect the presence of converted (PK resistant) PrP protein, a 96 well dot blot apparatus (Schleicher and Schuell) was set up with a sheet of 0.45 μm PVDF Immobilon-P (Millipore) membrane and each dot rinsed with 500 μL of TBS. Under vacuum, the lysed and PK-treated samples were added to the apparatus over the PVDF membrane and rinsed again with 500 μL of TBS. The PVDF membrane was then removed and covered with 3 M GdnSCN (Fluka) for 10 minutes at ambient temperature. The GdnSCN was removed by 5 PBS rinses and the membrane blocked in 5% (w/v) milk, 0.05% (v/v) Tween 20 (Sigma) in TBS (TBST-milk) for 30 minutes. An appropriate dilution of a monoclonal antibody 6B10, an IgG 2a reactive against mouse, hamster, elk, and sheep PrP in immunoblots and ELISA assays or 8 μg of purified 6H4 anti-PrP mouse monoclonal antibody (Prionics) in 15 mL TBST-milk was incubated with the membrane for 60 minutes. After rinsing with TBST, a solution of ˜500 ng of an alkaline phosphatase conjugated goat anti-mouse linked antibody (Zymed) in 15 mL TBST-milk was added for 45 minutes. After additional TBST rinsing, the membrane was treated with enhanced chemofluorescence agent (Amersham) for 10 minutes, allowed to dry, and then scanned using a Storm Scanner (Molecular Dynamics). The intensity of the PrPsc signal from each well was quantitated using ImageQuant software (Molecular Dynamics).

We first tested REP 2006 activity against both 22L and RML strains of mouse scrapie (see Table 1)

TABLE 1
Inhibition of PrP conversion by REP 2006 (n = 3)

| | | % PrP conversion relative to control | | | |
|---|---|---|---|---|---|
| | | Strain 22L | | Strain RML | |
| compound | conc. | Average | Std. Dev. | Average | Std. Dev. |
| REP 2006 | 10000 | 6.84 | 9.15 | −0.94 | 8.29 |
| (nM) | 1000 | −3.69 | 7.27 | 3.95 | 9.52 |
| | 500 | 5.60 | 11.73 | 3.74 | 14.16 |
| | 100 | 15.61 | 12.01 | 5.95 | 7.00 |
| | 50 | −4.33 | 7.54 | −6.43 | 8.63 |
| Alexafluor | 10 | 107.98 | 29.95 | 115.03 | 41.25 |
| (uM) | 1 | 95.58 | 29.16 | 125.70 | 24.14 |

To determine where the IC50 of REP 2006's anti-PrP conversion activity was, we repeated this test using lower concentrations of REP 2006 including a sheep strain of prion, Rov-9 (see Table 2)

TABLE 2
Inhibition of PrP conversion by REP
2006 (low conc. range, n = 3)

| | % PrP conversion relative to control | | | | | |
|---|---|---|---|---|---|---|
| | Strain 22L | | Strain RML | | Strain Rov-9 | |
| conc. | | Std. | | Std. | | Std. |
| (nM) | Average | Dev. | Average | Dev. | Average | Dev. |
| 500 | −0.69 | 0.23 | 2.04 | 0.28 | −0.35 | 5.69 |
| 100 | 1.19 | 0.86 | 1.79 | 1.69 | 29.55 | 12.40 |
| 50 | 2.45 | 1.89 | 5.15 | 2.24 | 55.66 | 21.05 |
| 10 | 48.54 | 11.72 | 87.35 | 17.16 | 69.22 | 21.45 |
| 5 | 62.41 | 2.31 | 89.72 | 9.51 | nt | nt |
| 1 | 67.57 | 12.38 | 100.49 | 6.38 | nt | nt |
| 0.5 | 90.42 | 11.31 | 92.45 | 11.29 | nt | nt |

nt = not tested

We then tested to see if PS-ODN randomer inhibition of PrP conversion was dependent on randomer size. For this experiment, we tested PS-ODN randomers of different sizes (see Table 3).

TABLE 3
Inhibition of PrP Conversion by PS-ODN Randomers (n = 3)

| | | % conversion relative to control | | | |
|---|---|---|---|---|---|
| | | Strain 22L | | Strain RML | |
| compound | conc. | Average | Std. Dev. | Average | Std. Dev. |
| REP 2006 | 100 | 1.00 | 4.25 | 3.73 | 1.44 |
| (nM) | 50 | 3.25 | 2.42 | 6.59 | 5.85 |
| | 10 | 105.15 | 7.58 | 121.70 | 5.53 |
| REP 2004 | 1000 | 2.04 | 2.42 | 6.49 | 5.10 |
| (nM) | 500 | 92.98 | 7.54 | 63.43 | 5.67 |
| | 100 | 88.28 | 17.19 | 91.10 | 12.51 |
| | 50 | 77.32 | 17.05 | 101.48 | 9.60 |
| | 10 | 70.22 | 9.99 | 97.60 | 9.88 |
| REP 2003 | 1000 | 69.97 | 3.87 | 79.05 | 3.61 |
| (nM) | 500 | 88.92 | 15.61 | 92.94 | 2.29 |
| | 100 | 80.06 | 7.54 | 91.45 | 11.83 |
| | 50 | 83.12 | 5.91 | 100.72 | 3.59 |
| | 10 | 86.97 | 4.90 | 96.10 | 7.15 |

These data show that PS-ODN randomers have a potent anti-PrP conversion activity against 22L, RML and Rov-9 strains of scrapie. This demonstrated potent activity of REP 2006 against scrapie strains from different animals. Moreover, this activity is dependent on the size of the PS-ODN randomer used, with REP 2003 (10 mer) inactive, REP 2004 (20 mer) mildly active and REP 2006 (40 mer) highly potent (IC50˜10 nM).

Thus, these data show that PS-ODN randomers are active against prion disease, and thus can be used in anti-prion therapy useful in the treatment of prion-based diseases in both humans (e.g., CJD), in animals (e.g., BSE, foot and mouth disease) and in the sterilization or prophylactic treatment of humans, animals and of blood and feed products which may be tainted by prions.

### EXAMPLE 2

### Tests for Determining if an Oligonucleotide Acts Predominantly by a Sequence Independent Mode of Action

An ON, e.g., ODN, in question shall be considered to be acting predominantly by a sequence independent mode of action if it meets the criterion of any one of the tests outlined below.

TEST #1—Effect of Partial Degeneracy on Anti-prion Efficacy

This test serves to measure the anti-prion activity of a particular ON sequence when part of its sequence is made degenerate. If the degenerate version of the ON having the same chemistry retains its activity as described below, is it deemed to be acting predominantly by a sequence independent mode of action. ONs will be made degenerate according to the following rule:
L_{ON}=the number of bases in the original ON
X=the number of bases on each end of the oligo to be made degenerate (but having the same chemistry as the original ON)
If L_{ON} is even, then X=L_{ON}/4
If L_{ON} is odd, then X=integer (L_{ON}/4)+1

Each degenerate base shall be synthesized according to any suitable methodology, e.g., the methodology described herein for the synthesis of PS-ON randomers.

The IC50 values shall be generated by a test of anti-prion efficacy accepted by the pharmaceutical industry. IC50 values shall be generated using a minimum of seven concentrations of compound, with three or more points in the linear range of the dose response curve. Using this test, the IC₅₀ of said ON shall be compared to its degenerate counterpart. If the IC₅₀ of the degenerate ON is less than 2-fold greater than the original ON for an ON of 25 bases and less, or is less than 10-fold greater than the original ON for ONs 26 bases or more (based on minimum triplicate measurements, standard deviation not to exceed 15% of mean) then the ON shall be deemed to be functioning predominantly by a sequence independent mode of action.

TEST #2—Comparison of Efficacy with Randomer

This test serves to compare the anti-prion efficacy of an ON with the anti-prion efficacy of a randomer ON of equivalent size and the same chemistry in the same prion disease.

The IC50 values shall be generated by a test of anti-prion efficacy accepted by the pharmaceutical industry. IC50 values shall be generated using a minimum of seven concentrations of compound, with three or more points in the linear range of the dose response curve. Using this test, the IC₅₀ of the ON shall be compared to an ON randomer of equivalent size and the same chemistry. If the IC₅₀ of the degenerate ON is less than 2-fold greater than the original ON for an ON of 25 bases and less, or is less than 10-fold greater than the original ON for ONs 26 bases or more (based on minimum triplicate measurements, standard deviation not to exceed 15% of mean) then the ON shall be deemed to be functioning predominantly by a sequence independent mode of action.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to provide oligonucleotides of various lengths and chemical modifications and/or various methods of administration can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

1. A method for the prophylaxis or treatment of a prion disease in a subject, comprising administering to a subject in need of such treatment a therapeutically effective amount of at least one pharmacologically acceptable oligonucleotide, wherein said oligonucleotide provides anti-prion activity.

2. The method of claim 1, wherein the anti-prion activity of said oligonucleotide occurs principally by a sequence independent mode of action.

3. The method of claim 1, wherein said at least one oligonucleotide comprises a mixture of at least two anti-prion randomers of different lengths.

4. The method of claim 1, wherein said oligonucleotide is at least 6 nucleotides in length and the sequence of said oligonucleotide is not complementary to any portion of a genomic sequence of said subject.

5. The method of claim 1, wherein said oligonucleotide is at least 6 nucleotides in length and the sequence of said oligonucleotide is complementary to a portion of a genomic sequence of said subject.

6. The method of claim 1, wherein said oligonucleotide is at least 6 nucleotides in length and at least a portion of the sequence of said oligonucleotide is complementary to a portion of a PrP gene.

7. The method of claim 1, wherein said oligonucleotide has an IC₅₀ for a prion of 1.0, μM or less.

8. The method of claim 1, wherein said oligonucleotide has an IC₅₀ for a prion of 0.1, μM or less.

9. The method of claim 1, wherein said oligonucleotide has an IC50 for a prion of 0.05 μM or less.

10. The method of claim 1, wherein said oligonucleotide has an IC50 for a prion of 0.01, μM or less.

11. The method of claim 1, wherein said oligonucleotide is at least 6 nucleotides in length.

12. The method of claim 1, wherein said oligonucleotide is at least 10 nucleotides in length.

13. The method of claim 1, wherein said oligonucleotide is at least 20 nucleotides in length.

14. The method of claim 1, wherein said oligonucleotide is at least 30 nucleotides in length.

15. The method of claim 1, wherein said oligonucleotide is at least 40 nucleotides in length.

16. The method of claim 1, wherein said oligonucleotide is at least 80 nucleotides in length.

17. The method of claim 1, wherein said oligonucleotide is at least 120 nucleotides in length.

18. The method of claim 1, wherein said oligonucleotide comprises at least one phosphodiester linkage.

19. The method of claim 1, wherein said oligonucleotide comprises at least one modification to its chemical structure.

20. The method of claim 1, wherein each said oligonucleotide comprises at least one phosphorothioated linkage.

21. The method of claim 1, wherein said oligonucleotide comprises at least one 2′-O methyl modification to the ribose moiety.

22. The method of claim 1, wherein said oligonucleotide comprises at least one 2′-modification to the ribose moiety.

23. The method of claim 1, wherein said oligonucleotide comprises at least one methylphosphonate linkage.

24. The method of claim 1, wherein each said oligonucleotide comprises at least one phosphorodithioated linkage.

25. The method of claim 1, wherein said oligonucleotide is a concatemer consisting of two or more oligonucleotide sequences joined by a linker.

26. The method of claim 1, wherein said oligonucleotide is linked or conjugated at one or more nucleotide residues, to a molecule modifying the characteristics of the oligonucleotide to obtain one or more characteristics selected from the group consisting of higher stability, lower serum interaction, higher cellular uptake, higher PrP interaction, an improved ability to be formulated for delivery, a detectable signal, higher anti-prion activity, better pharmacokinetic properties, specific tissue distribution, lower toxicity.

27. The method of claim 1, said oligonucleotide is linked or conjugated to a polyethylene glycol.

28. The method of claim 1, wherein said oligonucleotide is double stranded.

29. The method of claim 1, wherein said oligonucleotide is double or single stranded and comprises at least one base which is capable of hybridizing via non-Watson-Crick interactions.

30. The method of claim 1, wherein said oligonucleotide is double or single stranded and comprises at least one abasic moiety.

31. The method of claim 1, wherein said oligonucleotide comprises at least one Gquartet motif portion.

32. The method of claim 1, wherein said oligonucleotide comprises at least one CpG motif portion.

33. The method of claim 1, wherein at least a portion of the sequence of said oligonucleotide comprises polyA, polyC, polyG, polyT, polyAC, polyAG, polyAT, polyCG, polyCT, polyGT, polyU, polyAU, polyCU, or polyGU.

34. The method of claim 1, wherein at least a portion of the sequence of said oligonucleotide comprises two or more repeated sequences.

35. The method of claim 1, wherein said at least one oligonucleotide comprises a mixture of at least two different anti-prion oligonucleotides.

36. The method of claim 1, wherein said at least one oligonucleotide comprises a mixture of at least 10 different oligonucleotides.

37. The method of claim 1, wherein said at least one oligonucleotide comprises a mixture of at least 100 different oligonucleotides.

38. The method of claim 1, wherein said at least one oligonucleotide comprises a mixture of at least 1000 different oligonucleotides.

39. The method of claim 1, where said at least one oligonucleotide comprises a mixture of at least 106 different oligonucleotides.

40. The method of claim 35, wherein a plurality of said different oligonucleotides are at least 6 nucleotides in length.

41. The method of claim 35, wherein a plurality of said different oligonucleotides are at least 10 nucleotides in length.

42. The method of claim 35, wherein a plurality of said different oligonucleotides are at least 20 nucleotides in length.

43. The method of claim 35, wherein a plurality of said different oligonucleotides are at least 40 nucleotides in length.

44. The method of claim 35, wherein a plurality of said different oligonucleotides are at least 60 nucleotides in length.

45. The method of claim 35, wherein a plurality of said different oligonucleotides are at least 80 nucleotides in length.

46. The method of claim 35, wherein a plurality of said different oligonucleotides are at least 120 nucleotides in length.

47. A method for reducing prion activity in a biological material in vitro, comprising contacting said material with at least one anti-prion oligonucleotide.

48. The method of claim 47, wherein said biological material is animal blood.

49. The method of claim 47, wherein said biological material is an animal blood product.

50. The method of claim 47, wherein said biological material is a mammalian tissue.

51. The method of claim 47, wherein said biological material is a mammalian organ.

52. An anti-prion pharmaceutical composition comprising a therapeutically effective amount of at least one pharmacologically acceptable, anti-prion oligonucleotide; and a pharmaceutically acceptable carrier, wherein said composition is adapted for the treatment, control, or prevention of a prion disease.

53. The anti-prion pharmaceutical composition of claim 52, adapted for delivery by a mode selected from the group consisting of oral ingestion, enterally, inhalation, cutaneous injection, intraocular, subcutaneous injection, intramuscular injection, intraperitoneal injection, intrathecal injection, intraventricular, intracerebral injection, intratrachael injection, and intravenous injection.

54. The anti-prion pharmaceutical composition of claim 52, further comprising a delivery system.

55. The anti-prion pharmaceutical composition of claim 52, further comprising a liposomal formulation.

56. The anti-prion pharmaceutical composition of claim 54, wherein said delivery system or liposomal formulation targets specific cells or specific tissues.

57. The anti-prion pharmaceutical composition of claim 54, wherein said delivery system or liposomal formulation comprises at least one pegylated molecule.

58. The anti-prion pharmaceutical composition of claim 57, wherein said delivery system or liposomal formulation comprises an antibody.

59. The anti-prion pharmaceutical composition of claim 52, wherein said composition further comprises at least one other anti-prion drug in combination.

60. The anti-prion pharmaceutical composition of claim 52, wherein said composition further comprises a non-nucleotide polymer in combination.

61. The anti-prion pharmaceutical composition of claim 60, wherein said polymer is anionic.

62. A kit comprising at least one anti-prion oligonucleotide composition according to claim 52 in a labeled package, wherein a label or insert in said package indicates that said oligonucleotide can be used in treatment, control, or prevention of a prion disease.

63. The kit of claim 62, wherein said kit contains a mixture of at least two different oligonucleotides.

64. The kit of claim 62, wherein said kit is approved by a regulatory agency for use in humans.

65. The kit of claim 62, wherein said kit is approved by a regulatory agency for use in at least one non-human animal.
